(19) United States
(12) Patent Application Publication
(10) Pub. No.: US 2026/0111243 A1
(43) Pub. Date: Apr. 23, 2026

(54) Systems and Methods for Artificial Intelligence Based User Interface Configurations

(71) Applicant: SK Hynix NAND Product Solutions Corp. (dba Solidigm), Rancho Cordova, CA (US)

(72) Inventors: Caleb MCMILLAN, Forest Grove, OR (US); Matt A. YURDANA, Portland, OR (US); Rita H. WOUHAYBI, Portland, OR (US)

(21) Appl. No.: 18/919,215

(22) Filed: Oct. 17, 2024

(51) Int. Cl. G06F 9/451; A61B 5/16

(52) U.S. Cl. CPC G06F 9/451; A61B 5/165

(57) ABSTRACT

Some embodiments are directed to systems and methods that dynamically generate user interfaces according to a user's task, context, or current environment. In one aspect, a computer system includes one or more processors and memory. The computer system obtains a stream of sensor data from one or more sensors. The computer system generates a context attribute based on the stream of sensor data, the context attribute characterizing a condition of a physical environment or a user, The computer system, based on the context attribute, determines a layout configuration of a user interface to be presented to the user. The computer system dynamically renders the user interface for the user based on the layout configuration and displays the user interface on the display of the user interface.

### TECHNICAL FIELD

The present application generally relates to computer technology, and more particularly to, methods, systems, and non-transitory computer readable storage media for dynamically rendering user interfaces for a user according to a task, context, and/or current environment associated with the user.

### BACKGROUND

User interfaces enable a user to interact with software applications and perform tasks. A well-executed user interface facilitates effective interaction between a user and a program, application, or machine through clean designs, high responsiveness, and easy-to-read visuals.

### SUMMARY

A user interface can be applied to perform different tasks that involve a wide range of choices, contexts, understanding, and interactions from different users. In some instances, users with diverse personas interact with the same user interface. Using a warehousing application as an example, users of different personas can include a forklift operator who operates a forklift for moving goods from one location to another in a physical warehouse and reports products that may be damaged, a quality assessment (QA) engineer who assesses the product defects, and a claims inspector who fills out and submits claim forms for defective products. These personas can have a wide range of skillsets and/or job responsibilities, which can lead to the further broadening and complicating of a user interface.

The goal of an effective user interface is to make the user's experience easy and intuitive, while requiring minimum effort on the user's part to receive desired outcome. In accordance with some embodiments of this application described herein is a realization that a single user interface may not be able to accommodate a variety of contexts and tasks, cater to users with different personas, while also remaining simple and easy to use. Even for users with the same persona (e.g., forklift operators), user response to the same user interface can vary depending on circumstances such as a level of attentiveness of the user, and/or whether the user is interacting with the user interface during the day or at night. Further, in accordance with some embodiments of this application described herein is a realization that existing solutions rely on preset configurations or rules to configure and build a user interface and that these existing user interfaces are not capable of responding dynamically to different personas and changing context.

In view of the aforementioned reasons, there is a need for methods, systems, and non-transitory computer readable storage media for dynamically generating (e.g., on-the-fly, in real time, without user intervention) user interfaces for a user according to a task, context, and/or current environment associated with the user.

Some embodiments of the present disclosure are directed to methods, systems, and non-transitory computer readable storage media for dynamically generating user interfaces. In accordance with some embodiments of the present disclosure, a dynamic user interface generator utilizes an artificial intelligence (AI) system to generate, in real-time, a user interface design and layout for a user in accordance with user persona and the tasks the user needs to accomplish. In some embodiments, the AI system applies attributes of the user's job role, tasks, context, and current environment to learn the user interface design and layout that would produce the best user experience for the user in the present context of the user.

In accordance with some embodiments, the technical solutions disclosed advantageously distinguish over existing user interfaces or user interface builders by enabling capability that does not exist today. By dynamically generating user interfaces according to factors such as a user's persona, tasks, context, and current environment, the methods, systems, and user interfaces disclosed herein advantageously improve user-machine interaction, improve user experience, reduce a cognitive load of the user, and reduce a likelihood of user errors.

In one aspect, a method for generating user interfaces is implemented at a computer system having one or more processors and memory. The method includes obtaining a stream of sensor data from one or more sensors. The method includes generating a context attribute based on the stream of sensor data, the context attribute characterizing a condition of a physical environment or a user. The method includes, based on the context attribute, determining a layout configuration of a user interface to be presented to the user. The method includes dynamically rendering the user interface for the user based on the layout configuration. The method further includes displaying the user interface on the display of the user interface.

In some embodiments, generating the context attribute includes estimating a cognitive load of the user according to at least the stream of sensor data.

In some embodiments, generating the context attribute includes estimating a likelihood of user error in a task performed by the user according to at least the stream of sensor data.

In some embodiments, the method includes obtaining a predefined layout attribute. The layout configuration is determined based on the context attribute and the predefined layout attribute jointly. In some embodiments, the predefined layout attribute includes one or more of: credentials of the user, a work shift of the user, a current task performed by the user, a geographical location of the user, or a preferred language of the user.

In some embodiments, the method includes applying a context extraction model to process the stream of sensor data and generate the context attribute. In some embodiments, the method includes segmenting the stream of sensor data to form a plurality of sensor data segments based on a temporal window. The context extraction model is applied to process each sensor data segment. In some embodiments, the context extraction model includes a sensor feature extraction model and a context analysis model. Applying the context extraction model includes applying the sensor feature extraction model to extract a sensor feature vector based on each sensor data segment and applying the context analysis model to process the sensor feature vector and generate the context attribute.

According to another aspect of the present application, a computer system includes one or more processors and memory. The memory stores instructions that, when executed by the one or more processors, cause the computer system to perform any of the methods for dynamically generating user interfaces as disclosed herein.

According to another aspect of the present application, a non-transitory computer readable storage medium stores instructions configured for execution by a computer system that includes one or more processors and memory. The instructions, when executed by the one or more processors, cause the computer system to perform any of the methods for dynamically generating user interfaces as disclosed herein.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

The accompanying drawings, which are included to provide a further understanding of the embodiments, are incorporated herein, constitute a part of the specification, illustrate the described embodiments, and, together with the description, serve to explain the underlying principles.

FIG. 1 depicts a representative smart work environment, in accordance with some implementations.

FIG. 2 is an example operating environment in which a smart device interacts with a client device or a server system, in accordance with some implementations.

FIG. 3 is a block diagram illustrating a computer system of a smart work environment, in accordance with some implementations.

FIG. 4 is a block diagram of a machine learning system for training and applying data processing models using machine learning, in accordance with some embodiments.

FIG. 5A is a structural diagram of an example neural network applied to process work data in a data processing model, in accordance with some embodiments.

FIG. 5B is an example node in the neural network, in accordance with some embodiments.

FIG. 6 illustrates an exemplary workflow for dynamically rendering user interfaces, in accordance with some embodiments.

FIGS. 7A and 7B illustrate exemplary attributes that are input into a layout generation module, in accordance with some embodiments.

FIGS. 8A, 8B, and 8C illustrate exemplary user interfaces for different personas and tasks, in accordance with some embodiments.

FIG. 8D illustrates a previous view and an optimized view that is displayed in a user interface, in accordance with some embodiments.

FIGS. 9A to 9C provide a flowchart of an example method( for dynamically generating user interfaces, in accordance with some embodiments.

Like reference numerals refer to corresponding parts throughout the several views of the drawings.

### DETAILED DESCRIPTION

Reference will now be made in detail to specific embodiments, examples of which are illustrated in the accompanying drawings. In the following detailed description, numerous non-limiting specific details are set forth in order to assist in understanding the subject matter presented herein. But it will be apparent to one of ordinary skill in the art that various alternatives may be used without departing from the scope of the claims and the subject matter may be practiced without these specific details. For example, it will be apparent to one of ordinary skill in the art that the subject matter presented herein can be implemented on many types of electronic devices with digital video capabilities.

Various embodiments of the present disclosure are directed to dynamically generating user interfaces according to a user's task, context, or current environment. In accordance with some embodiments of the present disclosure, a computer system includes one or more processors, memory, and a display. The computer system obtains a stream of sensor data from one or more sensors. In some embodiments, the one or more sensors include environmental sensors for detecting ambient conditions of a physical environment. In some embodiments, the one or more sensors include user-facing sensors for detecting user expressions (e.g., whether a user is alert, stressed, confused) of a user associated with the physical environment and/or user interactions with devices (e.g., number of clicks, keystrokes, or positions of clicks). In some embodiments, the user is physically located in the physical environment. In some embodiments, the one or more sensors include wearable sensors that are worn by the user. The computer system generates a context attribute based on the stream of sensor data. The context attribute characterizes a condition of the physical environment or the user. In some embodiments, the context attribute relates (e.g., correlates or associates) the stream of sensor data with what a user needs in order to perform their tasks. In some embodiments, the computer system estimates (e.g., determines or predicts) a cognitive load of the user according to at least the stream of sensor data. In some embodiments, the computer system estimates a likelihood of user error in a task performed by the user according to at least the stream of sensor data. In some embodiments, the computer system applies a context extraction model to process the stream of sensor data and generate the context attribute. The computer system determines a layout configuration of a user interface to be presented to the user based on the context attribute. In some embodiments, the computer system applies a layout generation model or a predefined layout rule to process the context attribute and generate the layout configuration. The computer system dynamically renders (e.g., generates, in real time, without user intervention) the user interface for the user based on the layout configuration. The computer system displays the user interface on the display of the user interface. In some embodiments, after rendering the user interface, the computer system determines a layout quality metric, such as a time per task, a number of keystrokes per task, a cursor heatmap, an error rate, and a bounce rate, and adjusts the layout generation model or the predefined layout rule based on the layout quality metric.

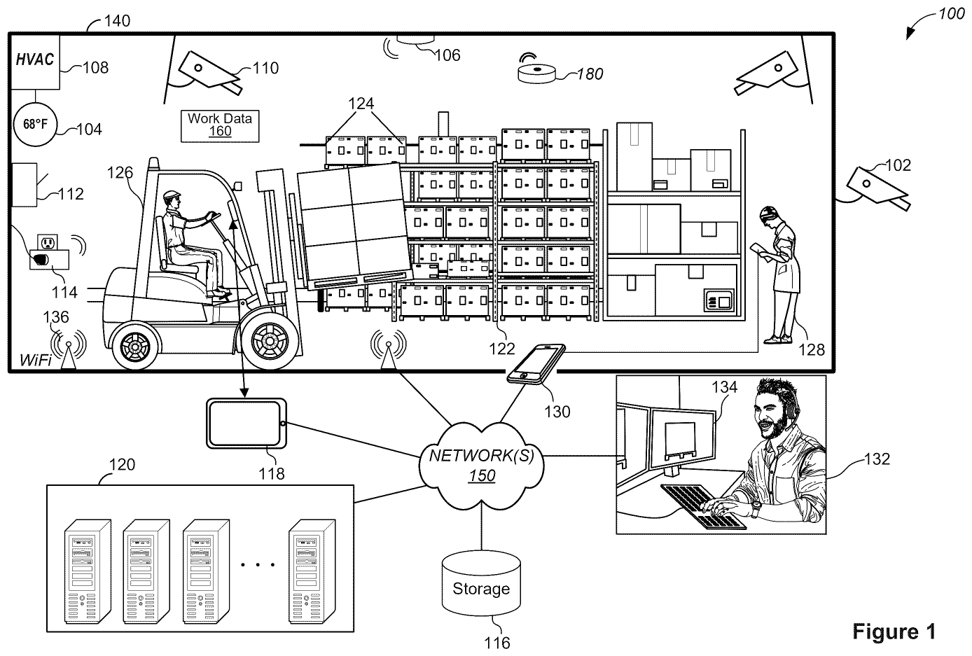

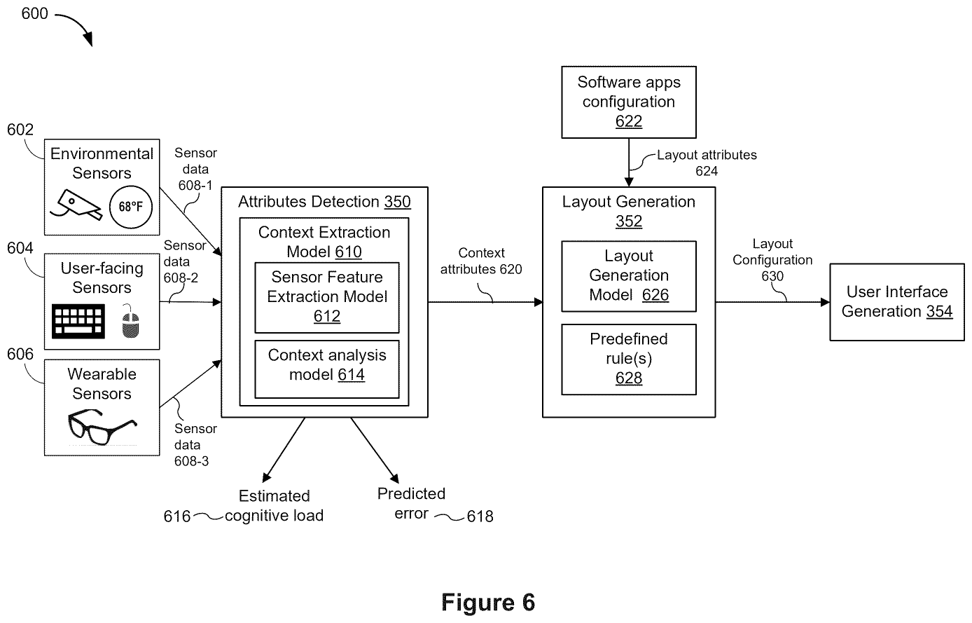

FIGS. 1-5B provide background exemplary sensor device networks and capabilities (e.g., machine learning based data processing capabilities) described herein, which are helpful in understanding the details of the embodiments described from FIG. 6 onward.

FIG. 1 depicts a representative smart work environment 100 in accordance with some implementations. The smart work environment 100 includes a structure 140, which may be used as a warehouse, factory, construction site, farm, laboratory, office space, retail store, hospital, and the like. For example, the structure 140 may be used as a distribution center, an e-commerce fulfillment center, an automobile assembly plant, an electronics manufacturing facility, a supermarket, or a retailer store. It will be appreciated that the structure 140 has an open floor plan, high ceilings, and support structures (e.g. columns or beams) and may include different functional areas designed for efficiency, safety, and scalability. Further, the smart work environment 100 may control and/or be coupled to devices outside of the actual structure 140. Indeed, several devices in the smart work environment 100 need not be physically within the structure 140. For example, a surveillance camera 102 may be located outside of the structure 140.

The depicted structure 140 may include a plurality of areas (e.g., storage areas, work areas) that may not be physically separated by walls. The depicted structure 140 may also include rooms (not shown) that are separated from the plurality of areas by walls. Devices may be mounted on, integrated with, and/or supported by a wall, a floor, a ceiling, or a support structure of the structure 140. Alternatively, devices may be mounted on, integrated with, and/or supported by an object (e.g., a shelf 122, a forklift 126) fixed or moveable in the structure 140.

In some implementations, the smart work environment 100 includes a plurality of devices, including intelligent, multi-sensing, network-connected devices, that integrate seamlessly with each other in a network 150 and/or with a central server system 120 or a cloud-computing system to provide a variety of useful smart work functions. The smart work environment 100 may include one or more surveillance cameras 102, one or more intelligent, multi-sensing, network-connected thermostats 104 (“smart thermostats”) and one or more intelligent, network-connected, multi-sensing hazard detection units 106 (“smart hazard detectors”). In some implementations, the smart thermostat 104 detects ambient climate characteristics (e.g., temperature and/or humidity) and controls an HVAC system 108 accordingly. The smart hazard detector 106 may detect the presence of a hazardous substance or a substance indicative of a hazardous substance (e.g., smoke, fire, and/or carbon monoxide). The surveillance cameras 102 may detect a person's or a vehicle's approach to or departure from the structure 140, identify and/or report any abnormal incidents, and/or control settings on a security system (e.g., to activate or deactivate the security system).

In some implementations, the smart work environment 100 includes one or more intelligent, multi-sensing, network-connected wall switches 112 (“smart wall switches”), along with one or more intelligent, multi-sensing, network-connected wall plug interfaces 114 (“smart wall plugs”). The smart wall switches 112 may detect ambient lighting conditions, detect room-occupancy states, and control a power and/or dim state of one or more lights. In some instances, smart wall switches 112 may also control a power state or speed of a fan, such as a ceiling fan. The smart wall plugs 114 may detect occupancy of a room or enclosure and control supply of power to one or more wall plugs (e.g., such that power is not supplied to the plug if nobody is present in the structure 140).

In some implementations, the smart work environment 100 includes a plurality of network-connected cameras 110 that are configured to provide video monitoring and security inside the structure 140. For example, the structure 140 is used as a warehouse, which is a bustling hub of activity, with neatly organized shelves 122 stretching high to accommodate an extensive inventory of product boxes 124. Each shelf 122 is carefully labeled and arranged to maximize space and ensure efficient access to goods. A forklift 126 may navigate the wide aisles with precision, lifting and moving boxes 124 from one location to another with a steady hum of its engine. The forklift 126 may include a computer device 118 for obtaining and updating information of the boxes 124 (e.g., box locations, weights, handling details). A worker 128 may check the stock levels on a handheld device 130, verifying the quantities and ensuring that inventory records match the physical stock. The air is filled with the sounds of the forklift's beeping and the occasional rustle of boxes as the warehouse maintains a routine of receiving, storing, and preparing products for distribution. A plurality of cameras 110 are distributed at different locations in the structure 140, and configured to capture static images or video clips monitoring activities of the forklift 126 and the worker 128.

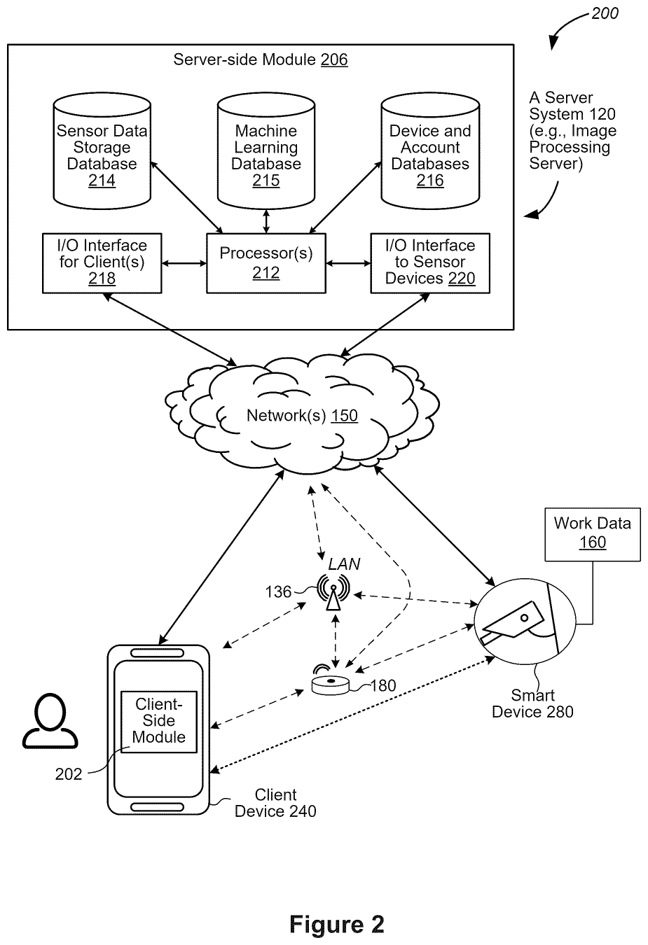

The devices 102-114 (e.g., collectively called smart devices 280 in FIG. 2) are examples of sensors and actuators that are disposed in the smart work environment 100 for collecting work data 160 (e.g., image data captured by cameras 110, temperature data captured by the smart thermostat 104). In some embodiments now shown, a variety of smart devices 280 are used to optimize efficiency and ensure smooth operations in the smart work environment 100. For example, radio frequency identification (RFID) sensors are employed to track products throughout the structure 140, ensuring that items are accurately located and inventoried. Proximity sensors may help robots and autonomous vehicles navigate safely by detecting obstacles and other machines. Infrared and optical sensors are used for barcode scanning, enabling quick identification of products. Additionally, pressure and weight sensors ensure that items are handled carefully and that shipping weights are accurate. Additional environmental sensors monitor conditions such as humidity to protect sensitive products. These technologies work together to create a highly automated and efficient smart work environment 100.

By virtue of network connectivity, one or more of the smart devices 280 may further allow a user to interact with the devices even if a user 132 is not proximate to the devices For example, the user 132 may communicate with a device using a computer device 134 (e.g., a desktop computer, laptop computer, a tablet computer, or other portable electronic device (e.g., a smartphone)). A webpage or application may be configured to receive communications from the user 132 and control the smart devices 280 based on the communications and/or to present information about the device's operation to the user 132. For example, the user 132 may view a current set point temperature for the smart thermostat 104 and adjust it using the computer device 134. The user 132 may review signature events captured by the camera 110 or adjust settings of the camera 110 using the computer device 134. The user 132 may be physically located within or outside the structure 140 during this remote communication.

As discussed above, users may control the smart thermostat 104 and other smart devices in the smart work environment 100 using a network-connected computer device 134. In some examples, a plurality of employees of a business entity associated with the structure 140 may register their devices 134 with the smart work environment 100. Such registration may be made at a central server 120 to authenticate the employees and/or the devices 134 as being associated with the structure 140 and to give permission to the employees to use the devices 134 to access the smart devices 280 in the structure 140. Employees may use their registered devices 134 to remotely control the smart devices 280 of the structure 140, e.g., when an employee is at work, on vacation, or at a separate office location. The employee may also use a registered device 134 (e.g., handheld device 130) to control the smart devices 280 when the employee is actually located inside the structure 140, such as when the employee is checking stocking in the warehouse.

In some implementations, in addition to containing processing and sensing capabilities, the devices 102, 104, 106, 108, 110, 112, and/or 114 (“the smart devices”) are capable of data communications and information sharing with other smart devices, a central server or cloud-computing system, and/or other devices that are network-connected. The required data communications may be carried out using any of a variety of custom or standard wireless protocols (e.g., IEEE 802.15.4, Wi-Fi, ZigBee, 6LoWPAN, Thread, Z-Wave, Bluetooth Smart, ISA100.11a, WirelessHART, or MiWi) and/or any of a variety of custom or standard wired protocols (e.g., CAT6 Ethernet or HomePlug), or any other suitable communication protocol.

In some implementations, the smart devices 280 serve as wireless or wired repeaters. For example, a first one of the smart devices communicates with a second one of the smart devices via a wireless router. The smart devices may further communicate with each other via a connection to one or more networks 150 such as the Internet. Through the one or more networks 150, the smart devices may communicate with a smart work server system 120 (also called a central server system and/or a cloud-computing system herein). In some implementations, the smart work server system 120 may include multiple server systems, each dedicated to data processing associated with a respective subset of the smart devices (e.g., a video server system may be dedicated to data processing associated with camera(s) 110). The smart work server system 120 may be associated with a manufacturer, support entity, or service provider associated with the smart devices 280. In some implementations, the smart work environment 100 relies on a dedicated hub device 180 to manage smart devices 280 located within the smart work environment 100, and a hub device server system associated with the hub device 180 serves as the server system 120.

In some implementations, a user is able to contact customer support using a smart device itself rather than needing to use other communication means, such as a telephone or Internet-connected computer. In some implementations, software updates are automatically sent from the smart work server system 120 to smart devices 280 (e.g., when available, when purchased, or at routine intervals). In some embodiments, the smart work environment 100 further includes a storage 116 for storing data related to the servers 120, smart devices 280, client devices 118, 130, and 134 (e.g., collectively called client device 240 in FIG. 2), and applications executed on the client devices. In some embodiments, the storage 116 includes a plurality of SSDs.

FIG. 2 is an example operating environment 100 in which a smart device 280 (e.g., cameras 110) interacts with a client device 240 (e.g., devices 118, 130, and 134 in FIG. 1) or a server system 120 (e.g., an image processing server), in accordance with some implementations. In the operating environment 200, the server system 120 provides data processing for monitoring and facilitating review of object location/motion associated with imaging device data streams (e.g., raw or processed work data 160) captured by multiple cameras 110 disposed in the structure 140. As shown in FIG. 2, the server system 120 may receive raw or processed work data 160 from smart devices 280 (standalone or integrated) located at various physical locations in the smart work environments 100. Each smart device 280 may be bound to one or more reviewer accounts, and the server system 120 may further process the received work data 160 to obtain information associated with the smart device 280 and the corresponding reviewer accounts. For a camera 110, the obtained information could be object locations, object movements, user gestures, and depth mapping. In some implementations, the server system 120 provides the information to client devices 240 associated with the reviewer accounts. In some implementations, the server system 120 uses the information to control a smart device 280 linked to the reviewer accounts.

In some implementations, the server system 120 is a dedicated image processing server that provides data processing services to cameras 110 and client devices 240 independently of other services provided by the server system 120.

In some implementations, each of the smart devices 280 captures work data 160 using signal detectors and sends the captured work data 160 to the server system 120 substantially in real time. In some implementations, each of the smart devices 280 includes a controller device (e.g., a smart device in which a camera 110 is integrated) that serves as an intermediary between the smart device 280 and the server system 120. The controller device receives the work data 160 from the one or more smart devices 280, optionally performs some preliminary processing on the work data 160, and sends the processed work data 160 to the server system 120 on behalf of the one or more smart devices 280 substantially in real time. In some implementations, each smart device 280 has its own on-board processing capabilities to perform some preliminary processing on the captured work data 160 before sending the processed work data 160 (along with metadata obtained through the preliminary processing) to the controller device and/or the server system 120. In some implementations, the client device 240 located in the smart work environment 100 functions as the controller device to at least partially process the captured work data 160.

In accordance with some implementations, each of the client devices 240 includes a client-side module 202. The client-side module 202 communicates with a server-side module 206 executed on the server system 120 through the one or more networks 150. The client-side module 202 provides client-side functionality for information monitoring, review processing, and communication with the server-side module 206. The server-side module 206 provides server-side functionality for event monitoring and review processing for any number of client-side modules 202, each residing on a respective client device 240. The server-side module 206 also provides server-side functionality for response processing and device control for any number of the smart devices 280.

In some implementations, the server-side module 206 includes one or more processors 212, a sensor data database 214, machine learning database 215, device and account databases 216, an I/O interface 218 to one or more client devices, and an I/O interface 220 to one or more smart devices 280. The I/O interface 218 to one or more clients facilitates the client-facing input and output processing for the server-side module 206. The device and account databases 216 store a plurality of profiles for reviewer accounts registered with the server system 120. A user profile includes account credentials for each reviewer account, and identifies one or more smart devices 280 linked to the reviewer account. In some implementations, the user profile of each reviewer account includes information related to capabilities, device characteristics, and lookup tables for the smart devices 280 linked to the reviewer account. The I/O interface 220 to one or more imaging devices facilitates communications with one or more smart devices 280 (standalone or integrated). The sensor data storage database 214 stores raw or processed work data 160 received from the smart devices 280 and associated information, as well as various types of metadata, such as device characteristics of signal emitters and detectors, lookup tables, modulation signals, and sampling rates. In some implementations, this data is used for generating additional information associated with each reviewer account. The machine learning database 215 stores data used by the server 120, the smart devices 280, or the client devices 240 to process the work data 160 collected by the smart devices 280 based on machine learning. For example, machine learning based data processing models and associated training data are stored in the machine learning database 215.

Client devices 240 include handheld computers, wearable computing devices, personal digital assistants (PDAs), tablet computers, laptop computers, desktop computers, cellular telephones, smart phones, enhanced general packet radio service (EGPRS) mobile phones, media players, navigation devices, game consoles, televisions, remote controls, point-of-sale (POS) terminals, vehicle-mounted computers, ebook readers, or a combination of any two or more of these data processing devices or other data processing devices.

Examples of the one or more networks 150 include local area networks (LANs) and wide area networks (WANs) such as the Internet. In some implementations, the one or more networks 150 are implemented using any known network protocol, including various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VoIP), Wi-MAX, or any other suitable communication protocol.

In some implementations, the server system 120 is implemented on one or more standalone data processing devices or a distributed network of computers. In some implementations, the server system 120 employs various virtual devices and/or services of third party service providers (e.g., third-party cloud service providers) to provide the underlying computing resources and/or infrastructure resources of the server system 120. In some implementations, the server system 120 includes handheld computers, tablet computers, laptop computers, desktop computers, or a combination of any two or more of these data processing devices or other data processing devices.

The server-client environment 200 shown in FIG. 2 includes both a client-side portion (e.g., the client-side module 202) and a server-side portion (e.g., the server-side module 206). The division of functionality between the client and server portions of operating environment 200 can vary in different implementations. Similarly, the division of functionality between the smart devices 280 and the server system 120 can vary in different implementations. In some implementations, the client-side module 202 is a thin-client that provides only user-facing input and output processing functions, and delegates other data processing functionality to a backend server (e.g., the server system 120). In some implementations, a smart device 280 is a simple data capturing device that continuously captures and streams work data 160 to the server system 120, with limited local preliminary processing of the data. Although many aspects of the present technology are described from the perspective of a computer system (e.g., system 300) as a whole, the corresponding actions performed by the client device 240 and/or the server system 120 would be apparent to those of skill in the art. Some aspects of the present technology may be described from the perspective of the client device or the server system, and the corresponding actions performed by the server system would be apparent to those of skill in the art. Furthermore, some aspects of the present technology may be performed by the server system 120, the client device 240, and the smart device 280 cooperatively.

It should be understood that the operating environment 200 that involves the server system 120, the client device 240, and the smart device 240 is merely an example. Many aspects of operating environment 200 are generally applicable in other operating environments in which a server system provides data processing for monitoring and facilitating review of data captured by other types of electronic devices.

The smart devices, the client devices, and the server system communicate with each other using the one or more communication networks 150. In an example smart work environment 100, two or more devices (e.g., the network interface device 136, the hub device 180, the client devices 240, and the smart devices 204) are located in close proximity to each other, such that they can be communicatively coupled in the same sub-network via wired connections, a WLAN, or a Bluetooth Personal Area Network (PAN). The Bluetooth PAN is optionally established based on classical Bluetooth technology or Bluetooth Low Energy (BLE) technology. In some implementations, each of the hub device 180, the client device 240, and the smart devices 204 are communicatively coupled to the networks 150 via the network interface device 136.

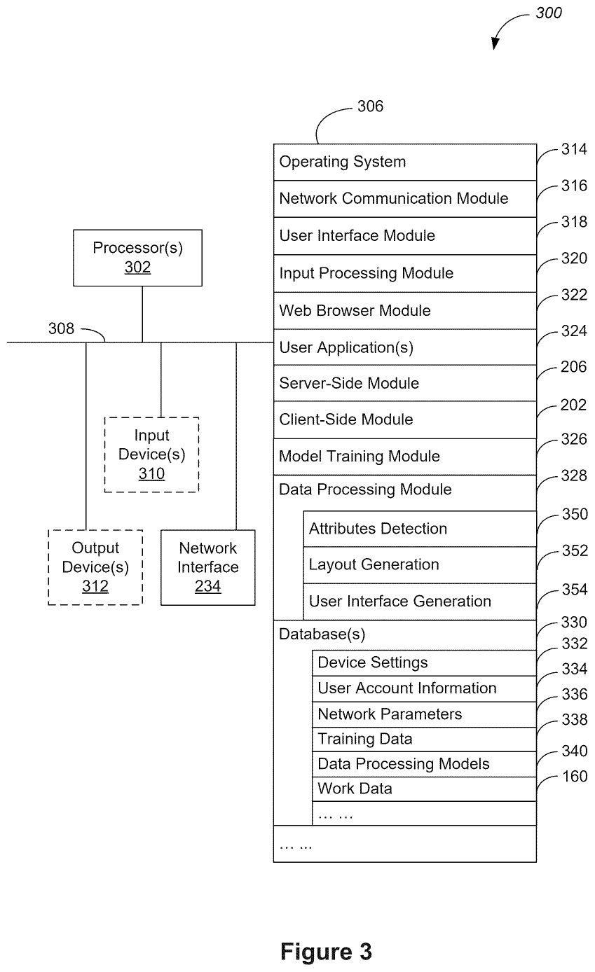

FIG. 3 is a block diagram illustrating a computer system 300 of a smart work environment 100 in accordance with some implementations. The computer system 300 includes a server 120, a client device 240 (e.g., computer device 118, 130, or 134 in FIG. 1), a smart device 280 (e.g., devices 102-114 in FIG. 1), a storage 116, or a combination thereof, and is configured to enable the smart work environment 100. The computer system 300 includes one or more processing units (CPUs) 302, one or more network interfaces 304, memory 306, and one or more communication buses 308 for interconnecting these components (sometimes called a chipset). In some implementations, the computer system 300 includes one or more input devices 310, which facilitate user input, such as a keyboard, a mouse, a voice-command input unit or microphone, a touch screen display, a touch-sensitive input pad, a gesture capturing camera, or other input buttons or controls. In some implementations, the computer system 300 uses a microphone and voice recognition or a camera and gesture recognition to supplement or replace the keyboard. In some implementations, the computer system 300 includes one or more cameras, scanners, or photo sensor units for capturing images. In some implementations, the computer system 300 includes one or more output devices 312, which enable presentation of user interfaces and display content, including one or more speakers and/or one or more visual displays.

The memory 306 includes high-speed random access memory, such as DRAM, SRAM, DDR RAM, or other random access solid state memory devices. In some implementations, the memory 306 includes non-volatile memory, such as one or more magnetic disk storage devices, one or more optical disk storage devices, one or more flash memory devices, or one or more other non-volatile solid state storage devices. In some implementations, the memory 306 includes one or more storage devices remotely located from the processing units 302. The memory 306, or alternatively the non-volatile memory within the memory 306, includes a non-transitory computer readable storage medium. In some implementations, the memory 306, or the non-transitory computer readable storage medium of the memory 306, stores the following programs, modules, and data structures, or a subset or superset thereof:
an operating system 314, which includes procedures for handling various basic system services and for performing hardware dependent tasks;
a network communication module 316, which connects the computer system 300 to other devices (e.g., various servers in the server system 120, a client device, or a smart device) via one or more network interfaces 304 (wired or wireless) and one or more networks 150, such as the Internet, other wide area networks, local area networks, metropolitan area networks, and so on;
a user interface module 318, which enables presentation of information (e.g., a graphical user interface for presenting applications, widgets, websites and web pages thereof, and/or games, audio and/or video content) at a client device 118, 130, and 134;
an input processing module 320 for detecting one or more user inputs or interactions from one of the one or more input devices 310 and interpreting the detected input or interaction;
a web browser module 322 for navigating, requesting (e.g., via HTTP), and displaying websites and web pages thereof, including a web interface for logging into a user account associated with a client device 140 or another electronic device, controlling the client or electronic device if associated with the user account, and editing and reviewing settings and data that are associated with the user account;
one or more user applications 324 for execution by the servers 120 (e.g., smart work applications, and/or other web or non-web based applications);
a server-side module 206, which communicates both with smart work environments 100 and with client-side modules 202 and includes a plurality of individual programs, procedures, modules, and/or objects for performing a variety of functions;
a client-side module 202, which communicates with the server-side module 206 in the smart work environment 100 and includes a plurality of individual programs, procedures, modules, and/or objects for performing a variety of functions;
model training module 326 for receiving training data and establishing one or more data processing models 340 for processing work data 160 (e.g., video, image, audio, or textual data) collected by the smart devices 280;
a data processing module 328 for processing work data 160 using data processing models 340, thereby identifying information contained in the work data 160, matching the work data 160 with other data, categorizing the work data 160, or synthesizing related work data 160; and
one or more databases 330 for storing at least data including one or more of:
   device settings 332 including common device settings (e.g., service tier, device model, storage capacity, processing capabilities, communication capabilities, etc.) of the one or more servers 120, client devices, or smart devices;
   user account information 334 for the one or more user applications 324, e.g., user names, security questions, account history data, user preferences, and predefined account settings;
   network parameters 336 for the one or more communication networks 150, e.g., IP address, subnet mask, default gateway, DNS server and host name;
   training data 338 for training one or more data processing models 340;
   data processing model(s) 340 for processing work data 160 (e.g., video, image, audio, or textual data) using deep learning techniques;
   work data 160 and associated results, where the work data 160 is processed using the data processing models 340 remotely at the server 120 or locally at the client device 240 to provide the associated results to be presented on the client devices or further processed.

In some implementations, the server-side module 106 acts as a control layer or API to the underlying functionality. In some implementations, the server-side module includes one or more of an emitter modulation module, a signal detection module, an object detection module, a location module, a movement module, a depth mapping module, and/or a gesture determination module for a smart device 280. Some implementations implement all of these features at a server system 120, some implementations implement all of these features at the camera 110, and some implementations distribute the functionality between the server 120 and the imaging device (e.g., based on efficiency considerations). In some implementations, the server-side module 206 includes a response processing module, which receives either raw unprocessed signals received at an camera 110 or signals that have been preprocessed by a local response processing module at the camera 110. The response processing module prepares the work data 160 (e.g., time of flight detection data) for use by the location module, the movement module, the depth mapping, and/or the gesture determination module. The server-side module 206 also includes an account administration module, which enables users to set up smart work environments 100 and to identify the smart devices 204 associated with the smart work environment 100.

In some embodiments, the data processing module 328 includes an attributes detection module 350, a layout generation module 352, and a user interface generation module 354. More details on the modules 350, 352, and 354 are discussed below with reference to FIGS. 6 to 9C.

Although many aspects of the present technology are described from the perspective of a computer system as a whole, the corresponding actions performed by the client device 240 and/or the server system 120 would be apparent to those of skill in the art. The server-side module 206 and the client-side module 202 are implemented at the server 120 and the client device 240, respectively. Each of the other modules 314-328 may be implemented in any of a server 120, a client device 240 (e.g., computer device 118, 130, or 134 in FIG. 1), a smart device 280 (e.g., devices 102-114 in FIG. 1), a storage 116, or a combination thereof.

Each of the above identified elements may be stored in one or more of the previously mentioned memory devices, and corresponds to a set of instructions for performing a function described above. The above identified modules or programs (e.g., sets of instructions) need not be implemented as separate software programs, procedures, modules, or data structures, and thus various subsets of these modules may be combined or otherwise rearranged in various implementations. In some implementations, the memory 306 stores a subset of the modules and data structures identified above. In some implementations, the memory 306 stores additional modules and data structures not described above.

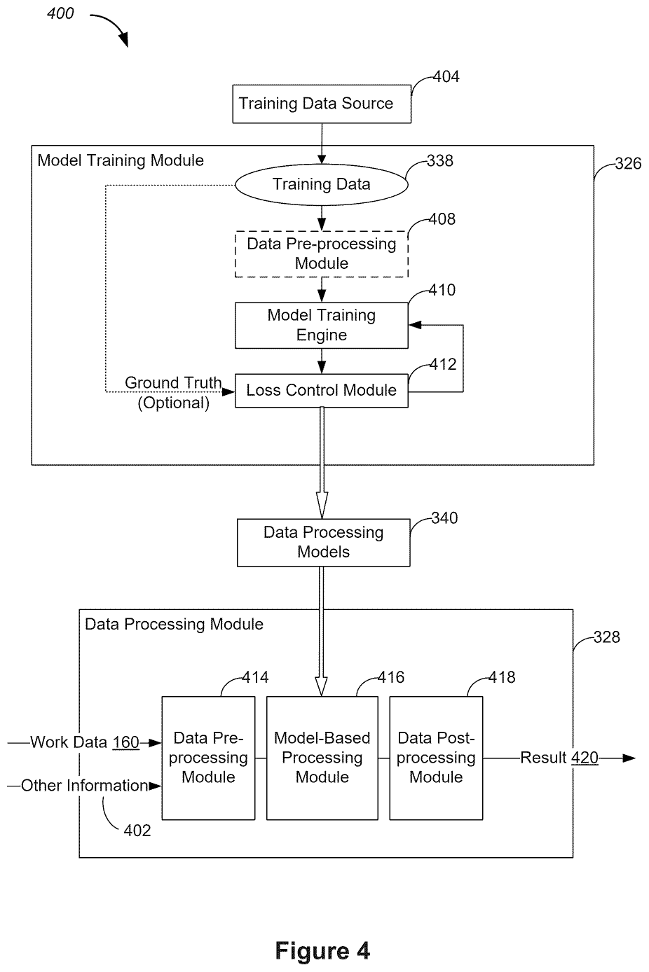

FIG. 4 is a block diagram of a machine learning system 400 for training and applying data processing models 340 using machine learning, in accordance with some embodiments. The machine learning system 400 includes a model training module 326 establishing one or more data processing models 340 and a data processing module 328 for processing data collected by smart devices 280 (e.g., cameras 110) using the data processing model 340. In some embodiments, both the model training module 326 (e.g., the model training module 326 in FIG. 3) and the data processing module 328 are located in the server 120, while a training data source 404 provides training data 338 to the server 120. In some embodiments, the training data source 404 is the data obtained from the smart devices 280, from another server 120, from storage 106, or from a client device. Alternatively, in some embodiments, the model training module 326 (e.g., the model training module 326 in FIG. 3) is located at a server 120, and the data processing module 328 is located in a smart device 280 or a client device 240. The server 120 trains the data processing models 328 and provides the trained models 340 to a smart device 280 or a client device 240 to process real-time work data 160 captured by the smart device 280.

In some embodiments, the training data 338 provided by the training data source 404 include a standard dataset (e.g., a set of work site images) widely used by engineers in an associated industry to train data processing models 340. In some embodiments, the training data 338 includes work data 160 and/or additional work site information, which is collected from one or more smart devices that will apply the data processing models 340 or collected from distinct smart devices that will not apply the data processing models 340. Further, in some embodiments, a subset of the training data 338 is modified to augment the training data 338. The subset of modified training data is used in place of or jointly with the subset of training data 338 to train the data processing models 340.

In some embodiments, the model training module 326 includes a model training engine 410, and a loss control module 412. Each data processing model 340 is trained by the model training engine 410 to process corresponding work data 160. Specifically, the model training engine 410 receives the training data 338 corresponding to a data processing model 340 to be trained, and processes the training data to build the data processing model 340. In some embodiments, during this process, the loss control module 412 monitors a loss function comparing the output associated with the respective training data item to a ground truth of the respective training data item. In these embodiments, the model training engine 410 modifies the data processing models 340 to reduce the loss, until the loss function satisfies a loss criteria (e.g., a comparison result of the loss function is minimized or reduced below a loss threshold). The data processing models 340 are thereby trained and provided to the data processing module 328 to process work data 160.

In some embodiments, the model training module 326 further includes a data pre-processing module 408 configured to pre-process the training data 338 before the training data 338 is used by the model training engine 410 to train a data processing model 340. For example, an image pre-processing module 408 is configured to format images in the training data 338 into a predefined image format. For example, the preprocessing module 408 may normalize the images to a fixed size, resolution, or contrast level. In another example, an image pre-processing module 408 extracts a region of interest (ROI) corresponding to a target area or object in each image or separates content of the target area or object into a distinct image.

In some embodiments, the model training module 326 uses supervised learning in which the training data 338 is labelled and includes a desired output for each training data item (also called the ground truth in some situations). In some embodiments, the desirable output is labelled manually by people or labelled automatically by the model training model 326 before training. In some embodiments, the model training module 326 uses unsupervised learning in which the training data 338 is not labelled. The model training module 326 is configured to identify previously undetected patterns in the training data 338 without pre-existing labels and with little or no human supervision. Additionally, in some embodiments, the model training module 326 uses partially supervised learning in which the training data is partially labelled.

In some embodiments, the data processing module 328 includes a data pre-processing module 414, a model-based processing module 416, and a data post-processing module 418. The data pre-processing modules 414 pre-processes work data 160 based on the type of the work data 160. In some embodiments, functions of the data pre-processing modules 414 are consistent with those of the pre-processing module 408, and convert the work data 160 into a predefined data format that is suitable for the inputs of the model-based processing module 416. The model-based processing module 416 applies the trained data processing model 340 provided by the model training module 326 to process the pre-processed work data 160. In some embodiments, the model-based processing module 416 also monitors an error indicator to determine whether the work data 160 has been properly processed in the data processing model 340. In some embodiments, the processed work data is further processed by the data post-processing module 418 to create a preferred format or to provide additional work information, associated with the smart work environment 100, which can be derived from the processed work data.

In some embodiments, work data 160 are supplemented with other information 402 (e.g., additional work site information, which is collected from one or more smart devices that will apply the data processing models 340 or collected from distinct smart devices that will not apply the data processing models 340). In some embodiments, the data processing module 328 uses the processed work data (e.g., result 420) to at least partially autonomously control an equipment or tool (e.g., forklift 126 in FIG. 1) that operates in the smart work environment 100. For example, the processed work data includes control instructions that are used by a control system (manned or unmanned) to drive the forklift 126. In some embodiments, the processed work data (e.g., result 420) is applied to at least partially autonomously control a robot operating on a vehicle assembly line or in an electronics manufacturing facility.

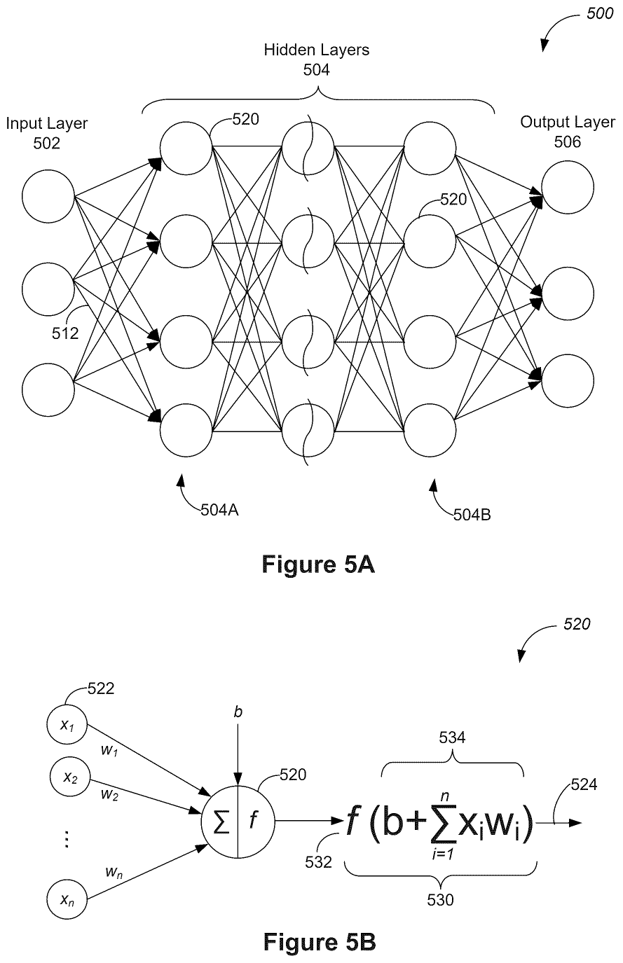

FIG. 5A is a structural diagram of an example neural network 500 applied to process work data in a data processing model 340, in accordance with some embodiments, and FIG. 5B is an example node 520 in the neural network 500, in accordance with some embodiments. It should be noted that this description is used as an example only, and other types or configurations may be used to implement the embodiments described herein. The data processing model 340 is established based on the neural network 500. A corresponding model-based processing module 416 applies the data processing model 340 including the neural network 500 to process work data 160 that has been converted to a predefined data format. The neural network 500 includes a collection of nodes 520 that are connected by links 512. Each node 520 receives one or more node inputs 522 and applies a propagation function 530 to generate a node output 524 from the one or more node inputs. As the node output 524 is provided via one or more links 512 to one or more other nodes 520, a weight w associated with each link 512 is applied to the node output 524. Likewise, the one or more node inputs 522 are combined based on corresponding weights w1, w2, w3, and w4 according to the propagation function 530. In an example, the propagation function 530 is computed by applying a non-linear activation function 532 to a linear weighted combination 534 of the one or more node inputs 522.

The collection of nodes 520 is organized into layers in the neural network 500. In general, the layers include an input layer 502 for receiving inputs, an output layer 506 for providing outputs, and one or more hidden layers 504 (e.g., layers 504A and 504B) between the input layer 502 and the output layer 506. A deep neural network has more than one hidden layer 504 between the input layer 502 and the output layer 506. In the neural network 500, each layer is only connected with its immediately preceding and/or immediately following layer. In some embodiments, a layer is a “fully connected” layer because each node in the layer is connected to every node in its immediately following layer. In some embodiments, a hidden layer 504 includes two or more nodes that are connected to the same node in its immediately following layer for down sampling or pooling the two or more nodes. In particular, max pooling uses a maximum value of the two or more nodes in the layer for generating the node of the immediately following layer.

In some embodiments, a convolutional neural network (CNN) is applied in a data processing model 340 to process work data (e.g., video and image data captured by cameras 110). The CNN employs convolution operations and belongs to a class of deep neural networks. The hidden layers 504 of the CNN include convolutional layers. Each node in a convolutional layer receives inputs from a receptive area associated with a previous layer (e.g., nine nodes). Each convolution layer uses a kernel to combine pixels in a respective area to generate outputs. For example, the kernel may be to a 3×3 matrix including weights applied to combine the pixels in the respective area surrounding each pixel. Video or image data is pre-processed to a predefined video/image format corresponding to the inputs of the CNN. In some embodiments, the pre-processed video or image data is abstracted by the CNN layers to form a respective feature map. In this way, video and image data can be processed by the CNN for video and image recognition or object detection.

In some embodiments, a recurrent neural network (RNN) is applied in the data processing model 340 to process work data 160. Nodes in successive layers of the RNN follow a temporal sequence, such that the RNN exhibits a temporal dynamic behavior. In an example, each node 520 of the RNN has a time-varying real-valued activation. It is noted that in some embodiments, two or more types of work data are processed by the data processing module 328, and two or more types of neural networks (e.g., both a CNN and an RNN) are applied in the same data processing model 340 to process the work data jointly.

The training process is a process for calibrating all of the weights wᵢ for each layer of the neural network 500 using training data 338 that is provided in the input layer 502. The training process typically includes two steps, forward propagation and backward propagation, which are repeated multiple times until a predefined convergence condition is satisfied. In the forward propagation, the set of weights for different layers are applied to the input data and intermediate results from the previous layers. In the backward propagation, a margin of error of the output (e.g., a loss function) is measured (e.g., by a loss control module 412), and the weights are adjusted accordingly to decrease the error. The activation function 532 can be linear, rectified linear, sigmoidal, hyperbolic tangent, or other types. In some embodiments, a network bias term b is added to the sum of the linear weighted combination 534 from the previous layer before the activation function 532 is applied. The network bias b provides a perturbation that helps the neural network 500 avoid over fitting the training data. In some embodiments, the result of the training includes a network bias parameter b for each layer.

FIG. 6 illustrates a workflow 600 for dynamically rendering user interfaces, in accordance with some embodiments. In some embodiments, the workflow 600 is implemented by the data processing module 328 that is described with respect to FIG. 3. In some embodiments, the data processing module 328 is an AI system. In some embodiments, the data processing module 328 includes an attributes detection module 350, a layout generation module 352, and a user interface generation module 354.

In some embodiments, the attributes detection module 350 obtains sensor data 608 from sensors of different sensor types. In the example of FIG. 6, the sensor data 608 includes sensor data 608-1 from environmental sensors 602, sensor data 608-2 from user-facing sensors 604, and sensor data 608-3 from wearable sensors 606. In some embodiments, the attributes detection module 350 obtains the sensors data 608-1, 608-2, and 608-3 simultaneously (or near simultaneously, such as within 0.1, 0.5, or 1 second) from the various sensors in real time or near real time, as the sensor data are being collected by the respective sensors. In some embodiments, the sensors data 608 include timestamps for identifying when a particular piece of data is collected.

In some embodiments, the sensors include environmental sensors 602 that are located in a physical environment. In some embodiments, the physical environment corresponds to an environment where a user interacting with a user interface is located. In some embodiments, the environmental sensors 602 can include one or more cameras (e.g., camera 110), one or more temperature sensors (e.g., thermostats 104) for detecting a temperature of the physical environment, one or more humidity sensors for detecting a level of humidity or relative humidity of the physical environment, one or more airflow sensors for measuring airflow in the physical environment, one or more pressure sensors for measuring ambient pressure, one or more vibration sensors for measuring vibrations from machineries of the physical environment, one or more gas sensors, one or more presence sensors, one or more moisture sensors for detecting a level of moisture in the physical environment, one or more light sensors for detecting an ambient light level in the physical environment, one or more radar sensors, one or more LiDAR sensors, and/or one or more motion sensors that are capable of detecting ambient conditions of the physical environment. In some embodiments, the ambient conditions (e.g., light level, temperature, or humidity) may affect a user's decisions. Examples of physical environments can include, and are not limited to, a warehouse, a storage facility, a distribution facility, a manufacturing site, an office space, or a traffic environment of a driving vehicle.

In some embodiments, the sensors include user-facing sensors 604 (e.g., peripheral sensors) that acquire user interaction data. Exemplary user-facing sensors 604 can include, and or not limited to, a camera, a motion sensor, a mouse device, a keyboard, a touch pad, and/or a microphone associated with the user. In some embodiments, the user interaction data includes keystroke activity, mouse click activity, eye movement of the user, and images of user expressions (e.g., whether a user is alert, confused, or distracted) as a user is interacting with the peripheral devices or data.

In some embodiments, the sensors include wearable sensors 606 that are disposed on wearable devices of a user of the physical environment. The wearable devices can include watches, eyeglasses, headphones, smart clothing, smart jewelry (e.g., rings) and/or fitness trackers. In some embodiments, the wearable sensors 606 are used for tracking physical activity and/or vital signs of the user.

In some embodiments, the sensors are not limited to the environmental sensors 602, user-facing sensors 604, or wearable sensors 606, but can include other sensors as well. For example, in a manufacturing environment, sensor data can be collected from different categories of sensors such as proximity sensors for welding, or voltage sensors, or current sensors, or resistance sensors for different units performing welding operations manufacturing. Sensor data from these sensors can also processed using the workflow 600 (e.g., by the attributes detection module to generate context attributes 620, as described below).

In some embodiments, the attributes detection module 350 processes the sensor data 608-1, 608-2, and 608-3, and generates context attributes 620 (e.g., inferred attributes, or attributes describing the environment and the user performing a task) based on at least a subset of the sensor data. In some embodiments, the attributes detection module 350 applies a context extraction model 610 (e.g., a neural network or data processing modules 340) to process the sensor data 608-1, 608-2, and 608-3 and generate the context attributes 620. In some embodiments, the context extraction model 610 includes a sensor feature extraction model 612 that is configured to extract sensor feature vectors based on the sensor data 608. In some embodiments, the context extraction model 610 includes a context analysis model 614 that is configured to process sensor feature vectors that are extracted by the sensor feature extraction model 612 and generate the context attributes 620.

In some embodiments, the context extraction model 610 is an AI system that is specifically trained to generate context attributes to be used by the layout generation module 352. For example, in some embodiments, the context extraction model 610 is trained to determine, from the timestamp-synchronized sensor data, how the ambient conditions (as measured by the environmental sensors 604) and user conditions (as measured by the wearable sensors 606) affect or influence user interaction data (as measured by the user-facing sensors 604).

In some embodiments, the attributes detection module 350 is configured to determine an estimated cognitive load 616 for a respective user according to at least a subset of the sensor data 608. For example, in some embodiments, the attributes detection module 350 is an AI model that is trained to output estimated cognitive loads for users by training on scenarios with known and quantified cognitive loads. In some embodiments where there is no existing training data, the attributes detection module 350 can be configured to initialize and output estimated cognitive loads using a pre-trained algorithm based on videos and closed caption text data scraped from the Internet and social media websites. In some embodiments, the attributes detection module 350 applies a transformer-based large multimodal model (LMM) that can understand and process different data modalities, including text, audio, video and/or sensory data, to output state-of-the-art prediction results on multi-modal data.

In some embodiments, the attributes detection module 350 is configured to determine a likelihood of user error (e.g., predicted error 618) in a task performed by a user according to at least a subset of the sensor data 608. In some embodiments, the attributes detection module 350 applies a transformer-based LMM that is configured to output prediction results on multi-modal data, to determine the predicted error 618.

With continued reference to FIG. 6, in some embodiments, the context attributes 620 generated by the attributes detection module 350 are input into a layout generation module 352 that is configured to determine (e.g., dynamically, in real time) a layout configuration 630 of a user interface to be presented to a user.

In some embodiments, the layout configuration 630 of a user interface includes a position, size, placement and/or arrangement of one or more buttons, menus, text fields, images, and other interactive elements of the user interface. In some embodiments, the layout configuration 630 includes a layout scheme of an element type (e.g., a button, a text, or an image) of an element of the user interface, a position of a user interface element in the user interface, a size of a user interface element in the user interface, an orientation of a user interface element in the user interface, a color encoding of a user interface element in the user interface, associated information, a navigation option, and a user action of each of a plurality of elements used to build the layout scheme.

In some embodiments, the layout generation module 352 obtains, from the configurations of one or more software applications (e.g., software apps configuration 622) executing on one or more devices of the smart environment and/or the user, layout attributes 624 (e.g., explicit attributes or explicit configurations), and generates the layout configuration 630 by combining information from both the context attributes 620 and the layout attributes 624. Examples of layout attributes 624 can include user credentials, user work shift (e.g., day shift or night shift), a current task performed by the user, a geographical location of the user, and/or a preferred language setting of the user.

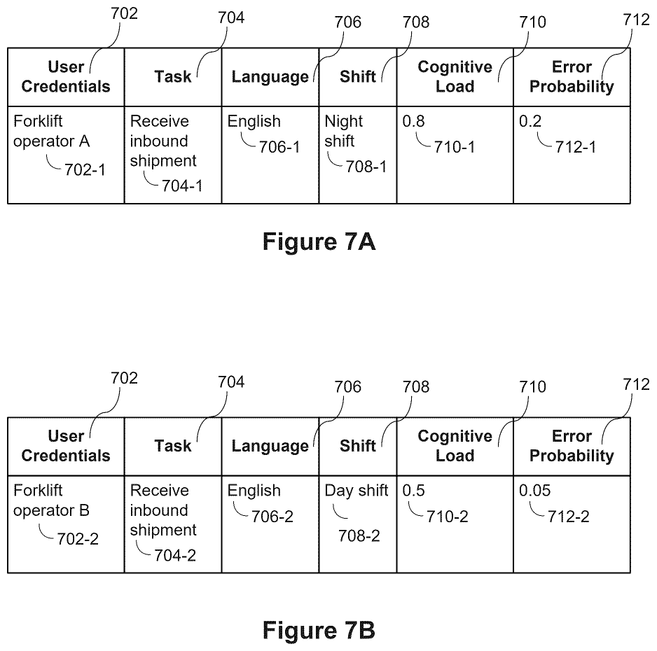

FIGS. 7A and 7B illustrate respective sets of exemplary attributes that are input into the layout generation module 352, in accordance with some embodiments, In some embodiments, a respective set of attributes include user credentials 702, task 704, language 706 (e.g., preferred language), shift 708, cognitive load 710 (e.g., estimated cognitive load 616), and error probability 712 (e.g., predicted error 618).

In the example of FIG. 7A, the user is forklift operator A who is using a user interface in a workstation that is used for managing warehouse operations. The user credentials (702-1), task (704-1), preferred language (706-1), and shift (708-1) are explicitly configured by the software application for managing warehouse operations. The cognitive load score 710-1 is determined (e.g., calculated) by the attributes detection module 350. In some embodiments, the attributes detection module 350 applies an LMM trained on camera and keystroke data. In some embodiments, when the attributes detection module 350 detects an increase in keystrokes and a stressed face on the user, it will generate (e.g., predict) a higher cognitive load score. The error probability value 712-1 is determined (e.g., calculated) by the attributes detection module 350. In some embodiments, the attributes detection module 350 applies an LMM trained on historical keystroke and environmental data to determine the error probability value. In this example, the attributes detection module 350 detects a significantly lower temperature than normal and determines that the night shift has an increased error rate. Accordingly, the attributes detection module 350 predicts a higher error rate, and reports an increased error probability, in accordance with some embodiments. The attributes 702-1, 704-1, 706-1, 708-1, 710-1, and 712-1 are input into the layout generation module for generating a layout configuration associated with this scenario.

FIG. 7B illustrates a scenario where, after eight hours of operation, there is a new user of the user interface identified as “Forklift Operator B” who is performing the same task 704-2 of receiving inbound shipments under a different set of environmental conditions. In this scenario, the set of attributes that are input into the layout generation module 352 includes attributes 702-2, 704-2, 706-2, 708-2, 710-2, and 712-2. In the example of FIG. 7B, the attributes detection module 350 detects a calm face and an average number of keystrokes. Therefore, the attributes detection module reports an average cognitive load score 710-2 that is lower than the cognitive load score 710-1. Additionally, based on historical keystroke data and task complexity, the attributes detection module 350 also reports a low error probability score 712-2. It should be apparent to one of ordinary skill in the art that although the example of FIGS. 7A and 7B describe a forklift operator, a similar analysis applies to other personas or subject matter experts. The personas can vary according to different industries. Other exemplary personas or subject matter experts can include welding experts, warehouse managers, or software developers.

Referring back to FIG. 6, in some embodiments, the layout generation module 352 applies a layout generation model 626 (e.g., a neural network, an AI model, or data processing models 340) to process the context attributes 620 and the layout attributes 624, and generate the layout configuration 630. In some embodiments, the layout generation module 352 applies one or more predefined rules 628 to process the context attributes 620 and the layout attributes 624, and generate the layout configuration 630. For example, the one or more predefined rules 628 can include a first rule to position a user interface element slightly leftward when the environmental sensors 602 register a lower temperature, or a second rule to change a contrast and/or brightness setting for text fields of a user interface when the user is a day-shift operator versus a night-shift operator.

With continued reference to FIG. 6, in some embodiments, the layout configuration 630 that is generated by the layout generation module 352 is fed into a user interface generation module 354. In some embodiments, the user interface generation module 354 is part of the data processing module 328. In some embodiments, the user interface generation module 354 is a user interface generation component of an existing application. In some embodiments, the user interface generation module 354 is configured to automatically render the user interface for the user based on the layout configuration 630 provided by the layout generation module 352. In some embodiments, the user interface generation module 354 causes display of the user interface on a display device (e.g., client device 240, or devices 118, 130, and 134 in FIG. 1). In some embodiments, the user interface generation module 354 is configured to modify an existing user interface that is displayed on a display device by magnifying or highlighting relevant or critical components, obfuscating irrelevant or distracting components, and/or creating an overlay to change the design of the UI.

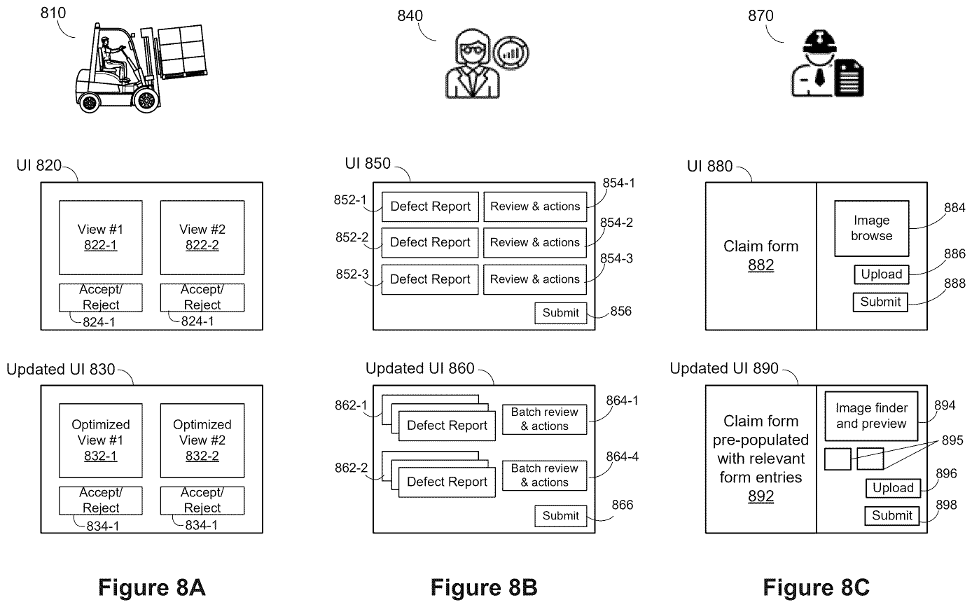

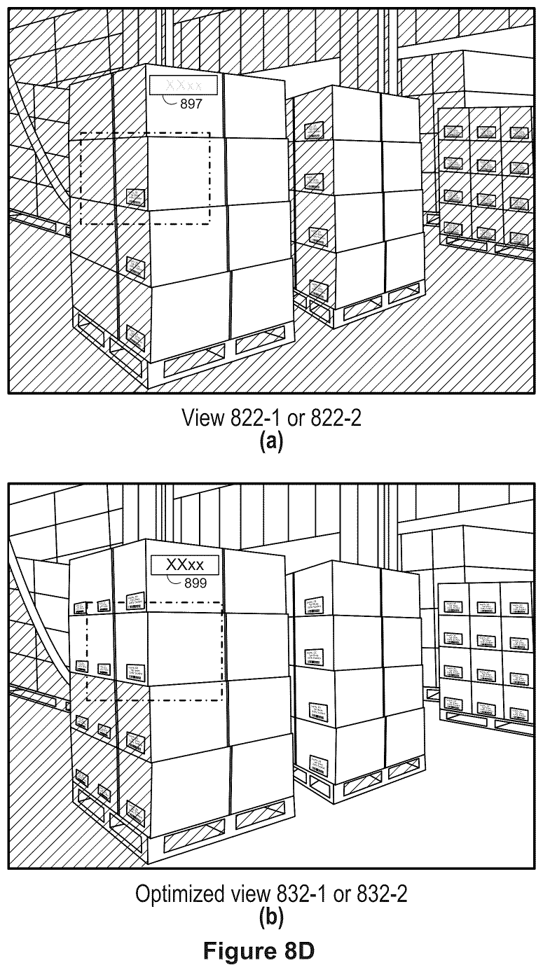

FIGS. 8A, 8B, and 8C illustrate exemplary user interfaces for different personas and tasks for a warehousing application, in accordance with some embodiments.

In some embodiments, the personas include a forklift operator 810 who operates a forklift, receives inbound shipments in a physical warehouse, and reports products that may be damaged. FIG. 8A illustrates a user interface 820 that is used by the forklift operator 810, in accordance with some embodiments. The user interface 820 includes views 822-1 and 822-2 corresponding to two different camera angles of the receiving area. The user interface 820 also includes accept/reject buttons 824-1 and 824-2 that, when selected by the forklift operator 810, provides instructions or feedback regarding defective shipments.

FIG. 8A illustrates an updated user interface 830 that is generated and rendered in accordance with the workflow 600, in accordance with some embodiments. For example, in some embodiments, the workflow 600 applies camera data and estimates a cognitive load of the forklift operator 810 and would update the views from views 822-1 and 822-2 to optimized views 832-1 and 832-2. In some embodiments, compared to views 822, the optimized views 832 provide improved brightness or contrast levels, making the views (e.g., images) easier to see, less straining for a user's eyes, and reduces a cognitive load on the forklift operator. FIG. 8D(a) illustrates view 822-1 or view 822-2 whereas FIG. 8D(b) illustrates optimized view 832-1 or optimized view 832-2, in accordance with some embodiments. The views in the example of FIGS. 8D(a) and 8D(b) are images of stacks of boxes in a warehouse. The views 822-1 and 822-2 in FIG. 8D(a) are darker and appear to have lower contrast whereas the optimized views 832-1 and 832-2 in FIG. 8D9B) are brighter and appear to have higher contrast, and therefore are more optimized for viewing. A comparison of these figures show that the barcode labels 899 on the boxes in optimized views 832-1 or 832-2 are easier to read compared to the barcode labels 897 in the views 822-1 or 822-2 due to higher contrast.

In some embodiments, the personas include a warehouse manager 840 who reviews the defect reports that are generated for an entire shift. FIG. 8B illustrates a user interface 850 that the warehouse manager 840 interacts with, in accordance with some embodiments. The user interface 850 includes of a list of defect reports 852 (e.g., defect report 852-1, 852-2, and 852-3), In some embodiments, a respective defect report 852 can include a thumbnail image of the defective item received, along with actions to be performed on the report (e.g., submit report as-is, include more details on the defective items, decline to submit report). In some embodiments, and as illustrated in FIG. 8B, the user interface 850 includes a set of review and action items 854 (e.g., review and actions 854-1, 854-2, and 854-3) corresponding to a respective defect report 852. In some instances, the warehouse manager 840 reviews the list of reports 852 and associated review and action items 854 to ensure the necessary defect reports are being filed, and hits submit button 856 on the user interface 850 to submit the defect reports 852 to a quality controller (QC) expert 870.

FIG. 8B illustrates an updated user interface 860 that is generated and rendered in accordance with the workflow 600, in accordance with some embodiments. In some embodiments, the attributes detection module 350 or layout generation module 352 analyzes image and/or tabular data in each defect report and groups together reports that are similar (e.g., having similar product defects). The updated user interface 860 is configured to presents defect reports as batches, such as defect report batch 1 862-1 and defect report batch 2 862-2 as illustrated in FIG. 8B. The updated user interface 860 also displays, for each defect report batch, a respective affordance 864 (e.g., affordance 864-1 and affordance 864-2) that enables warehouse manager 840 to act on a respective batch of defect reports. Once the warehouse manager 840 completes their review of the batches of defect reports, the warehouse manager 840 can click the submit button 866 to submit the reports. In some embodiments, the layout scheme of the updated user interface 860 advantageously reduces processing time by enabling batch processing of reports with similar defects, reduces a cognitive load of the warehouse manager, and reduces the likelihood of errors.

In some embodiments, the personas include QC expert 870 who fills out and submits claim forms for defective products. FIG. 8C illustrates a user interface 880 that the QC expert 870 interacts with, in accordance with some embodiments. In some embodiments, the user interface 880 displays a claim form 882 to be completed, an image browse affordance 884 that, when selected by the QC expert 870 (e.g., user), enables the QC expert 870 to browse images to be included in the claims form 882. In some embodiments, the user interface 880 also displays an upload affordance 886 that enables additional images to be uploaded and a submit affordance 888 that, when selected, causes the claim form 882 to be submitted.

FIG. 8C illustrates an updated user interface 890 that is generated and rendered in accordance with the workflow 600, in accordance with some embodiments.

In some embodiments, the updated user interface 890 displays a claim form that has been pre-populated with certain relevant form entries 892 (e.g., the claim form includes auto-filled information such as shipper ID based on the barcode shown on the image). In some embodiments, the updated user interface 890 displays an image finder and preview region 894 and thumbnail images 895 of suggested images (e.g., from camera data) to include in the claim form. In some embodiments, the thumbnails images 895 are selected for display on the updated user interface 890 based on an analysis (e.g., by the attributes detection module 350 or layout generation module 352) of image data and/or tabular data from defect reports and historical user data of the QC expert 870. In some embodiments, the updated user interface 890 displays an upload affordance 896 that enables additional images to be uploaded. In some embodiments, the updated user interface 890 displays a submit affordance 898 that, when selected, causes the claim form 892 to be submitted.

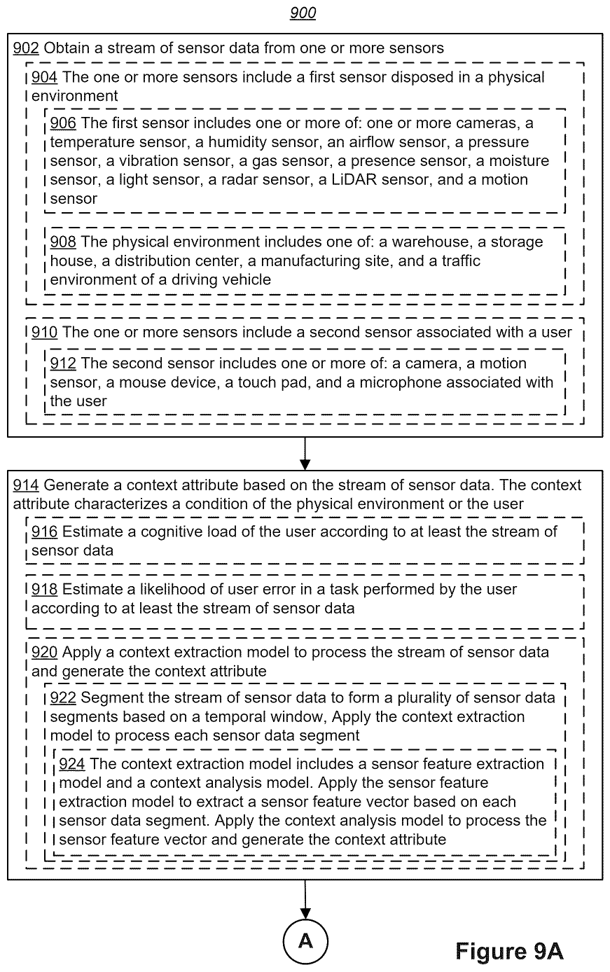

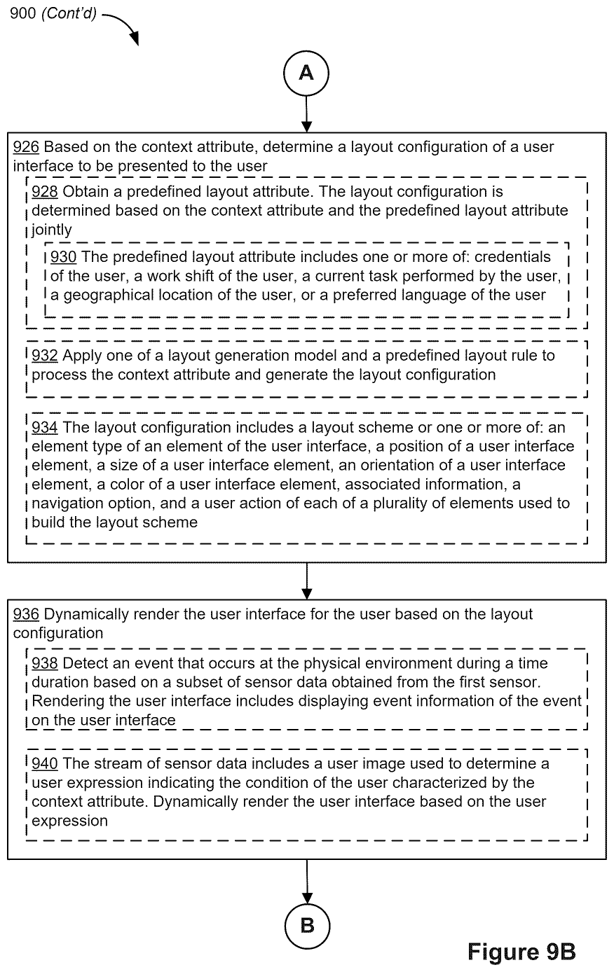

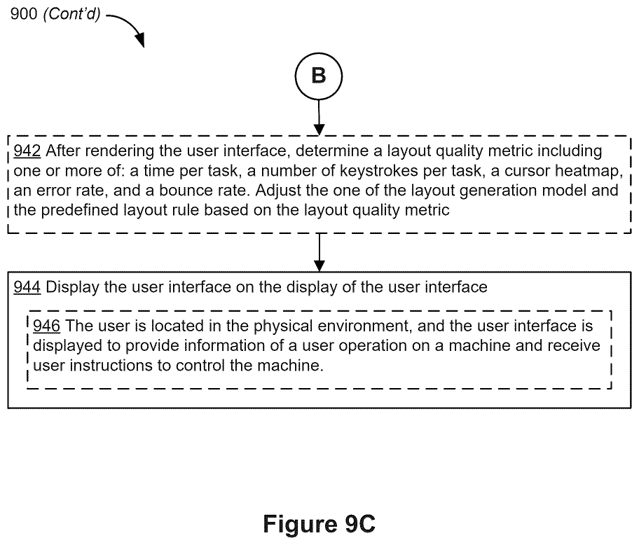

FIGS. 9A to 9C provide a flowchart of an example method 900 for dynamically generating user interfaces processing data, in accordance with some embodiments. The method 900 is performed at a computer system (e.g., computer system 300).

The computer system includes one or more processors (e.g., processor(s) 302 in FIG. 3) and memory (e.g., memory 306). In some embodiments, the memory stores one or more programs or instructions configured for execution by the one or more processors. In some embodiments, the operations shown in FIGS. 1, 2, 4, 5A, 5B, 6, 7A, 7B 8A, 8B, 8C, and 8D correspond to instructions stored in the memory or other non-transitory computer-readable storage medium. The computer-readable storage medium may include a magnetic or optical disk storage device, solid state storage devices such as Flash memory, or other non-volatile memory device or devices. In some embodiments, the instructions stored on the computer-readable storage medium include one or more of: source code, assembly language code, object code, or other instruction format that is interpreted by one or more processors. Some operations in the method 900 may be combined. The order of some operations may be changed.

Referring to FIG. 9A, the computer system obtains (operation 902) a stream of sensor data (e.g., sensors data 608-1, 608-2, and 608-3) from one or more sensors. In some embodiments, the one or more sensors include environmental sensors (e.g., environmental sensors 602) for detecting ambient conditions of the physical environment. In some embodiments, the one or more sensors include user-facing sensors (e.g., user-facing sensors 604) for detecting user expressions (e.g., whether a user is alert, stressed, confused, or distracted) and user interactions with devices (e.g., number of clicks, keystrokes, positions of clicks, etc.). In some embodiments, the one or more sensors include wearable sensors (e.g., wearable sensors 606) that are worn by a user of the environment. In some embodiments, the wearable sensors 606 collect the vitals of the user and correlate the vital signs with visual cues (e.g., obtained from the user-facing sensors 604) for better predictions. For example, when a user's facial expression shows frustration, it can be corelated with variations in the user's vitals, such as a higher pulse rate. In some instances where the user does not demonstrate visual cues, the user's vital signs can provide good indicators of the user's state of mind.

In some embodiments, the one or more sensors include (operation 904) a first sensor (e.g., environmental sensors 602) disposed in a physical environment.

In some embodiments, the first sensor includes (operation 906) one or more of: one or more cameras, a temperature sensor, a humidity sensor, an airflow sensor, a pressure sensor, a vibration sensor, a gas sensor, a presence sensor, a moisture sensor, a light sensor, a radar sensor, a LiDAR sensor, and a motion sensor.

In some embodiments, the physical environment includes (operation 908) one of: a warehouse, a storage house, a distribution center, a manufacturing site, and a traffic environment of a driving vehicle.

In some embodiments, the one or more sensors include (operation 910) a second sensor associated with the user (e.g., user-facing sensors 604 or wearable sensors 606). In some embodiments, the second sensor includes (operation 912) one or more of: a camera, a motion sensor, a mouse device, a touch pad, and a microphone associated with the user.

The computer system generates (operation 914) a context attribute (e.g., context attributes 620) based on the stream of sensor data. The context attribute characterizes a condition of a physical environment or a user. In some embodiments, the context attribute relates the sensor data to what a user needs to perform their tasks. In some embodiments, the context attribute includes an estimation of a cognitive load of a user (e.g., estimated cognitive load 616). In some embodiments, the context attribute includes an estimation of an error probability of a task (e.g., predicted error 618) performed by the user.

In some embodiments, generating the context attribute includes estimating (operation 916) (e.g., determining or predicting) a cognitive load of the user (e.g., estimated cognitive load 616) according to at least the stream of sensor data.

In some embodiments, generating the context attribute includes estimating (operation 918) (e.g., determining or predicting) a likelihood of user error (e.g., predicted error 618) in a task performed by the user according to at least the stream of sensor data.

In some embodiments, the computer system applies (operation 920) a context extraction model (e.g., context extraction model 610, data processing models 340) to process the stream of sensor data and generate the context attribute.

In some embodiments, the computer system segments (operation 922) the stream of sensor data to form a plurality of sensor data segments based on a temporal window, and applies the context extraction model to process each sensor data segment.

In some embodiments, the context extraction model includes a sensor feature extraction model (e.g., sensor feature extraction model 612) and a context analysis model (e.g., context analysis model 614). In some embodiments, the computer system applies (operation 924) the sensor feature extraction model to extract a sensor feature vector based on each sensor data segment. In some embodiments, the computer system applies (operation 924) the context analysis model to process the sensor feature vector and generate the context attribute.

Referring to FIG. 9B, the computer system, based on the context attribute, determines (operation 926) (e.g., via layout generation module 352) a layout configuration (e.g., layout configuration) of a user interface to be presented to the user.

In some embodiments, the computer system obtains (operation 928) a predefined layout attribute (e.g., layout attributes 624 or explicit attributes), and determines the layout configuration based on the context attribute and the predefined layout attribute jointly. This is illustrated in FIG. 6.

In some embodiments, the predefined layout attribute includes (operation 930) one or more of: credentials of the user, a work shift of the user (e.g., day shift or night shift) (e.g., shift 708), a current task (e.g., task 704) performed by the user, a geographical location of the user, or a preferred language (e.g., language 706) of the user. In some embodiments, the predefined layout attribute includes a persona of a user, a role or job function of the user, and/or user credentials (e.g., user credentials 702).

In some embodiments, the computer system applies (operation 932) a layout generation model (e.g., layout generation model 626 or data processing models 340) and/or a predefined layout rule (e.g., predefined rule(s) 628) to process the context attribute and generate the layout configuration, to determine the layout configuration.

In some embodiments, the layout configuration includes (operation 934) a layout scheme or one or more of: an element type of an element of the user interface, a position of a user interface element, a size of a user interface element, an orientation of a user interface element, a color of a user interface element, associated information, a navigation option, and a user action of each of a plurality of elements used to build the layout scheme.

The computer system dynamically renders (operation 936) the user interface for the user based on the layout configuration. In some embodiments, dynamically rendering the user interface for the user includes generating, by the computer system in real time, without user intervention, the user interface for the user.

In some embodiments, the computer system detects (operation 938) an event that occurs at the physical environment during a time duration based on a subset of sensor data obtained from the first sensor. Rendering the user interface includes displaying event information of the event on the user interface. An example of an event that can occur in a warehouse setting is a forklift accident that may result in damaged products. In this example, the user interface can display information of the accident and an identification of products that may be damaged.

In some embodiments, the stream of sensor data includes (operation 940) a user image used to determine a user expression indicating the condition of the user characterized by the context attribute (e.g., whether the user appears alert, attentive, confident, confused, distracted, stressed, or inattentive). In some embodiments, the computer system dynamically renders the user interface based on the user expression. In some embodiments, the user interface is used to facilitate user control of a machine.

Referring to FIG. 9C, in some embodiments, after rendering the user interface, the computer system determines (operation 942) a layout quality metric including one or more of: a time per task, a number of keystrokes per task, a cursor heatmap, an error rate, and a bounce rate. In some embodiments, the computer system adjusts the one of the layout generation model and the predefined layout rule based on the layout quality metric.

The computer system displays (operation 944) the user interface on the display of the user interface.

In some embodiments, the user is located in the physical environment. The user interface is (operation 946) displayed to provide information of a user operation on a machine and receive user instructions to control the machine.

It should be understood that the particular order in which the operations in FIGS. 9A to 9C have been described are merely exemplary and are not intended to indicate that the described order is the only order in which the operations could be performed. One of ordinary skill in the art would recognize various ways to dynamically generating user interfaces as described herein. Additionally, it should be noted that details of other processes described herein with respect to other figures (e.g., FIGS. 1-8D) are also applicable in an analogous manner to method 900 described above with respect to FIGS. 9A to 9C. For brevity, these details are not repeated here.

Turning on to some example embodiments:
(A1) In accordance with some embodiments, a method for generating user interfaces is performed at a computer system having one or more processors and memory. The method includes (i) obtaining a stream of sensor data from one or more sensors; (ii) generating a context attribute based on the stream of sensor data, the context attribute characterizing a condition of a physical environment or a user; (iii) based on the context attribute, determining a layout configuration of a user interface to be presented to the user; (iv) dynamically rendering the user interface for the user based on the layout configuration; and (v) displaying the user interface on the display of the user interface
(A2) In some embodiments of A1, generating the context attribute includes estimating a cognitive load of the user according to at least the stream of sensor data.
(A3) In some embodiments of A1 or A2, generating the context attribute includes estimating a likelihood of user error in a task performed by the user according to at least the stream of sensor data
(A4) In some embodiments of any of A1-A3, the method includes obtaining a predefined layout attribute, wherein the layout configuration is determined based on the context attribute and the predefined layout attribute jointly.
(A5) In some embodiments of A4, the predefined layout attribute includes one or more of: credentials of the user, a work shift of the user, a current task performed by the user, a geographical location of the user, or a preferred language of the user.
(A6) In some embodiments of any of A1-A5, the one or more sensors include a first sensor disposed in a physical environment. The method includes detecting an event that occurs at the physical environment during a time duration based on a subset of sensor data obtained from the first sensor, wherein rendering the user interface includes displaying event information of the event on the user interface.
(A7) In some embodiments of any A6, the first sensor includes one or more of: one or more cameras, a temperature sensor, a humidity sensor, an airflow sensor, a pressure sensor, a vibration sensor, a gas sensor, a presence sensor, a moisture sensor, a light sensor, a radar sensor, a LiDAR sensor, and a motion sensor.
(A8) In some embodiments of A6 or A7, the physical environment includes one of: a warehouse, a storage house, a distribution center, a manufacturing site, and a traffic environment of a driving vehicle.
(A9) In some embodiments of any of A1-A8, the one or more sensors include a second sensor associated with the use. The second sensor includes one or more of: a camera, a motion sensor, a mouse device, a touch pad, and a microphone associated with the user.
(A10) In some embodiments of any of A1-A9, the user is located in the physical environment, and the user interface is displayed to provide information of a user operation on a machine and receive user instructions to control the machine. The stream of sensor data includes a user image used to determine a user expression indicating the condition of the user characterized by the context attribute. The user interface is dynamically rendered based on the user expression.
(A11) In some embodiments of any of A1-A10, the method includes applying a context extraction model to process the stream of sensor data and generate the context attribute.
(A12) In some embodiments of A11, the method includes segmenting the stream of sensor data to form a plurality of sensor data segments based on a temporal window, wherein the context extraction model is applied to process each sensor data segment.
(A13) In some embodiments of A12, the context extraction model includes a sensor feature extraction model and a context analysis model. Applying the context extraction model includes applying the sensor feature extraction model to extract a sensor feature vector based on each sensor data segment and applying the context analysis model to process the sensor feature vector and generate the context attribute.
(A14) In some embodiments of any of A1-A13, determining the layout configuration of the user interface includes applying one of a layout generation model and a predefined layout rule to process the context attribute and generate the layout configuration.
(A15) In some embodiments of A14, the method includes, after rendering the user interface, determining a layout quality metric including one or more of: a time per task, a number of keystrokes per task, a cursor heatmap, an error rate, and a bounce rate. The method includes adjusting the one of the layout generation model and the predefined layout rule based on the layout quality metric.
(A16) In some embodiments of any of A1-A15, the layout configuration includes a layout scheme of one or more of: an element type of an element of the user interface, a position of a user interface element, a size of a user interface element, an orientation of a user interface element, a color of a user interface element, associated information, a navigation option, and a user action of each of a plurality of elements used to build the layout scheme
(B1) In accordance with some embodiments, a computer system includes one or more processors and memory. The memory stores one or more programs for execution by the one or more processors. The one or more programs include instructions for performing the method of any of A1-A16.
(C1) In accordance with some embodiments, a non-transitory computer-readable storage medium stores one or more programs for execution by one or more processors. The one or more programs include instructions for performing the method of any of A1-A16.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,”depending on the context.

It is also to be appreciated that while the terms user may be used to refer to the person or persons acting in the context of some particularly situations described herein, these references do not limit the scope of the present teachings with respect to the person or persons who are performing such actions. Importantly, while the identity of the person performing the action may be germane to a particular advantage provided by one or more of the implementations, such identity should not be construed in the descriptions that follow as necessarily limiting the scope of the present teachings to those particular individuals having those particular identities.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

As used herein, the phrase “based on” does not mean “based only on,” unless expressly specified otherwise. In other words, the phrase “based on” describes both “based only on” and “based at least on.”

As used herein, the term “exemplary” means “serving as an example, instance, or illustration,” and does not necessarily indicate any preference or superiority of the example over any other configurations or implementations.

As used herein, the term “and/or” encompasses any combination of listed elements. For example, “A, B, and/or C” includes the following sets of elements: A only, B only, C only, A and B without C, A and C without B, B and C without A, and a combination of all three elements, A, B, and C.

The foregoing description, for purpose of explanation, has been described with reference to specific implementations. However, the illustrative discussions above are not intended to be exhaustive or to limit the invention to the precise forms disclosed. Many modifications and variations are possible in view of the above teachings. The implementations were chosen and described in order to best explain the principles of the invention and its practical applications, to thereby enable others skilled in the art to best utilize the invention and various implementations with various modifications as are suited to the particular use contemplated.

What is claimed is:

1. A method for generating user interfaces, comprising:
at a computer system having one or more processors, memory, and a display:
obtaining a stream of sensor data from one or more sensors;
generating a context attribute based on the stream of sensor data, the context attribute characterizing a condition of a physical environment or a user;
based on the context attribute, determining a layout configuration of a user interface to be presented to the user;
dynamically rendering the user interface for the user based on the layout configuration; and
displaying the user interface on the display of the user interface.

2. The method of claim 1, wherein generating the context attribute includes estimating a cognitive load of the user according to at least the stream of sensor data.

3. The method of claim 1, wherein generating the context attribute includes estimating a likelihood of user error in a task performed by the user according to at least the stream of sensor data.

4. The method of claim 1, further comprising:
obtaining a predefined layout attribute, wherein the layout configuration is determined based on the context attribute and the predefined layout attribute jointly.

5. The method of claim 4, wherein the predefined layout attribute includes one or more of: credentials of the user, a work shift of the user, a current task performed by the user, a geographical location of the user, or a preferred language of the user.

6. The method of claim 1, wherein the one or more sensors include a first sensor disposed in a physical environment, the method further comprising:
detecting an event that occurs at the physical environment during a time duration based on a subset of sensor data obtained from the first sensor, wherein rendering the user interface includes displaying event information of the event on the user interface.

7. The method of claim 6, wherein the first sensor includes one or more of: one or more cameras, a temperature sensor, a humidity sensor, an airflow sensor, a pressure sensor, a vibration sensor, a gas sensor, a presence sensor, a moisture sensor, a light sensor, a radar sensor, a LiDAR sensor, and a motion sensor.

8. The method of claim 6, wherein the physical environment includes one of: a warehouse, a storage house, a distribution center, a manufacturing site, and a traffic environment of a driving vehicle.

9. The method of claim 1, wherein the one or more sensors include a second sensor associated with the user, and the second sensor includes one or more of: a camera, a motion sensor, a mouse device, a touch pad, and a microphone associated with the user.

10. The method of claim 1, wherein:
the user is located in the physical environment, and the user interface is displayed to provide information of a user operation on a machine and receive user instructions to control the machine;
the stream of sensor data includes a user image used to determine a user expression indicating the condition of the user characterized by the context attribute; and
the user interface is dynamically rendered based on the user expression.

11. The method of claim 1, further comprising:
applying a context extraction model to process the stream of sensor data and generate the context attribute.

12. The method of claim 11, further comprising:
segmenting the stream of sensor data to form a plurality of sensor data segments based on a temporal window, wherein the context extraction model is applied to process each sensor data segment.

13. The method of claim 12, wherein the context extraction model includes a sensor feature extraction model and a context analysis model, and applying the context extraction model further comprises:
applying the sensor feature extraction model to extract a sensor feature vector based on each sensor data segment; and
applying the context analysis model to process the sensor feature vector and generate the context attribute.

14. The method of claim 1, wherein determining the layout configuration of the user interface further includes applying one of a layout generation model and a predefined layout rule to process the context attribute and generate the layout configuration.

15. The method of claim 14, further comprising:
after rendering the user interface, determining a layout quality metric including one or more of: a time per task, a number of keystrokes per task, a cursor heatmap, an error rate, and a bounce rate; and
adjusting the one of the layout generation model and the predefined layout rule based on the layout quality metric.

16. The method of claim 1, wherein the layout configuration includes a layout scheme of one or more of: an element type of an element of the user interface, a position of a user interface element, a size of a user interface element, an orientation of a user interface element, a color of a user interface element, associated information, a navigation option, and a user action of each of a plurality of elements used to build the layout scheme.

17. A computer system, comprising:
one or more processors; and
memory storing one or more programs for execution by the one or more processors, the one or more programs further comprising instructions for:
obtaining a stream of sensor data from one or more sensors;
generating a context attribute based on the stream of sensor data, the context attribute characterizing a condition of a physical environment or a user;
based on the context attribute, determining a layout configuration of a user interface to be presented to the user;
dynamically rendering the user interface for the user based on the layout configuration; and
displaying the user interface on the display of the user interface.

18. The computer system of claim 17, wherein the instructions for generating the context attribute include instructions for estimating a cognitive load of the user according to at least the stream of sensor data.

19. A non-transitory computer-readable storage medium, storing one or more programs for execution by one or more processors, the one or more programs further comprising instructions for:
obtaining a stream of sensor data from one or more sensors;
generating a context attribute based on the stream of sensor data, the context attribute characterizing a condition of a physical environment or a user;
based on the context attribute, determining a layout configuration of a user interface to be presented to the user;
dynamically rendering the user interface for the user based on the layout configuration; and
displaying the user interface on the display of the user interface.

20. The non-transitory computer-readable storage medium of claim 19, wherein the instructions for generating the context attribute include instructions for estimating a likelihood of user error in a task performed by the user according to at least the stream of sensor data.
